(19) United States
(12) Patent Application Publication
(10) Pub. No.: US 2015/0320498 A1
(43) Pub. Date: Nov. 12, 2015

(54) CHROMIUM ION-DOPED LASER APPARATUS FOR MEDICAL APPLICATION AND OPERATION METHOD THEREOF

(71) Applicant: Electronics and Telecommunications Research Institute, Daejeon (KR)

(72) Inventors: Won Bae CHO, Daejeon (KR); Moon Youn JUNG, Daejeon (KR); Dong Hoon SONG, Daejeon (KR)

(21) Appl. No.: 14/668,884

(22) Filed: Mar. 25, 2015

(30) Foreign Application Priority Data
May 9, 2014 (KR) 10-2014-0055244

(51) Int. Cl. A61B 18/20; A61B 19/00; H01S 3/105; H01S 3/16; H01S 3/094

(52) U.S. Cl. CPC A61B 18/20; H01S 3/1691; H01S 3/094; H01S 3/105; A61B 19/40; A61B 2018/204; A61B 2018/2025; A61B 2017/00141

(57) ABSTRACT

Provided is a chromium ion-doped laser apparatus for medical application and a method of operating the laser apparatus, the apparatus including a laser beam generating unit to generate a laser beam, a converting unit to convert a wavelength of the generated laser beam to be a set wavelength, and an emitting unit to emit the laser beam having the converted wavelength to an object.

### CROSS-REFERENCE TO RELATED APPLICATION

This application claims the priority benefit of Korean Patent Application No. 10-2014-0055244, filed on May 9, 2014, in the Korean Intellectual Property Office, the disclosure of which is incorporated herein by reference.

### BACKGROUND

1. Field of the Invention

Embodiments of the present invention relate to a laser apparatus for safely eliminating an abnormal tissue while minimizing an adverse effect on a normal tissue in a body by emitting a mid-infrared laser beam having a high fat or water absorbent property, and an operation method thereof.

2. Description of the Related Art

A laser apparatus may be used to eliminate an unnecessary amount of fat by emitting a laser beam to the fat in a body.

A general laser apparatus used for a laser lipolysis may emit a laser beam having a wavelength of 930 nanometers (nm) or 1064 nm. Since the wavelength of the emitted laser beam is relatively short, the laser beam may have a low fat absorbent property, for example, less than “1”.

Due to the low fat absorbent property, a high energy laser beam may need to be emitted to efficiently eliminate the fat in the body. However, the high energy laser beam may cause a burn injury on the body.

A laser apparatus using an optical parametric oscillator (OPO) light source may generate laser beams having wavelengths of 1980 nm and 2300 nm corresponding to a laser beam having a relatively long wavelength. However, when an energy, for example, a power for one of the laser beams is determined, an energy for another laser beam may be correspondingly determined and thus, an output power for each of the laser beams may not be adjusted separately. Also, in the laser apparatus based on the OPO light source, wavelength conversion may be determined based on an OPO crystal or a wavelength of a pump light source and thus, performing a necessary wavelength adjustment may be difficult.

### SUMMARY

An aspect of the present invention provides a laser apparatus for emitting a mid-infrared laser beam having a high fat or water absorbent property using a chromium ion-doped laser crystal, thereby safely eliminating an abnormal tissue while minimizing an effect on a normal tissue of a body, and a method of operating the laser apparatus.

Another aspect of the present invention also provides a method and apparatus for selectively outputting a continuous laser beam or a pulsed laser beam, or adjusting a wavelength of a laser beam in response to a laser mode change request, thereby emitting an appropriate laser beam based on a situation or a purpose.

According to an aspect of the present invention, there is provided a laser apparatus including a laser beam generating unit to generate a laser beam, a converting unit to convert a wavelength of the generated laser beam to be a set wavelength, and an emitting unit to emit the laser beam having the converted wavelength to an object.

According to another aspect of the present invention, there is also provided a method of operating a laser apparatus, the method including generating a laser beam, converting a wavelength of the generated laser beam to be a set wavelength, and emitting the laser beam having the converted wavelength to an object.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a block diagram illustrating an example of a laser apparatus according to an embodiment of the present invention;

FIG. 2 is a diagram illustrating another example of a laser apparatus according to an embodiment of the present invention; and

FIG. 3 is a flowchart illustrating a method of operating a laser apparatus according to an embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

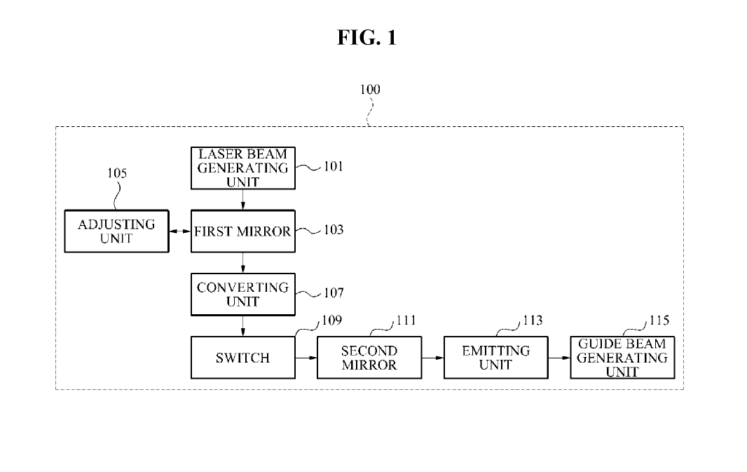

FIG. 1 is a block diagram illustrating an example of a laser apparatus 100 according to an embodiment of the present invention. In the present disclosure, the terms “laser” and “laser beam” may be used interchangeably.

Referring to FIG. 1, the laser apparatus 100 according to an embodiment of the present invention may include a laser beam generating unit 101, an adjusting unit 105, a converting unit 107, a switch 109, an emitting unit 113, and a guide beam generating unit 115. The laser apparatus 100 may include at least two mirrors, which may cause a change in a position of each element, for example, the adjusting unit 105 and the switch 109.

The laser beam generating unit 101 may generate a laser beam. The laser beam generating unit 101 may be, for example, a pump laser device corresponding to a laser diode (LD) available at a low cost. The laser beam generating unit 101 may also be an optical fiber laser device or a solid-state laser device. The laser beam generating unit 101 may generate a laser beam having a wavelength between 1300 nanometers (nm) and 2100 nm through which an absorbent property of the converting unit 107, for example, a mid-infrared laser beam crystal, is manifested. In this example, the laser beam generating unit 101 may be a laser beam doped with erbium (Er) ions, thulium (Tm) ions, holmium (Ho) ions, and the like.

A first mirror 103 may receive the generated laser beam, transfer the received laser beam to the converting unit 107 such that a wavelength of the laser beam is converted, transfer the laser beam having the converted wavelength to the adjusting unit 105, reflect the laser beam of which the wavelength is adjusted by the adjusting unit 105, and retransfer the reflected laser beam to the converting unit 107.

Based on a wavelength adjustment value input by a user, the adjusting unit 105 may receive the laser beam of which the wavelength converted by the converting unit 107 from the first mirror 103 and adjust the converted wavelength. In this example, the adjusting unit 105 may adjust the wavelength to be within an output range, for example, from 1700 nm to 3500 nm, of the converting unit 107.

The adjusting unit 105 may include, for example, an etalon, a wave plate, a birefringent filter, a grating, a prism, and the like.

The converting unit 107 may convert the wavelength of the received laser beam to be a set wavelength, and output the laser beam having a wavelength, for example, between 1700 nm and 3500 nm. The converting unit 107 may be, for example, a laser crystal doped with chromium, for example, chromium (II) (Cr²⁺), ions. In this example, the laser crystal may be provided in a cylindrical shape or a hexahedral shape, and fixed to a holder having a high thermal conductivity. To enhance a thermal conductivity, the laser crystal may be covered with a substance such as indium (In), and then fixed to the holder.

The switch 109 may receive the laser beam of which the wavelength is converted by the converting unit 107 and, in response to a laser mode change request, output the received laser beam through transformation to one of a continuous laser beam and a pulsed laser beam, which is related to the laser mode change request.

In this instance, when the continuous laser beam is output, the switch 109 may be disposed in an external area of a laser resonator (not shown). When the pulsed laser beam is output, the switch 109 may be disposed in an internal area of the laser resonator. For example, when the laser apparatus 100 is used for a medical purpose, the pulsed laser beam may be output and thus, the switch 109 may be disposed in the internal area of the laser resonator.

The switch 109 may include, for example, a saturable absorber, a pockels cell, an electro-optic modulator, an acoustic-optic modulator, and the like.

A second mirror 111 may be a mirror performing a function of a laser output mirror. The second mirror 111 may receive the laser beam output from the switch 109, reflect a significant portion of the received laser beam, penetrate another portion of the received laser beam, and transfer the penetrated portion to the emitting unit 113.

The emitting unit 113 may receive the laser beam output from the second mirror 111 and emit the received laser beam to an object, for example, a fat cell. Here, the emitting unit 113 may include a cannula used to emit a laser beam to a body, for example, an abnormal tissue of the body corresponding to the object.

In this instance, the emitting unit 13 may emit a laser beam having a fat or water absorbent property greater than or equal to a set reference value. The laser beam emitted from the laser apparatus 100 may have, for example, an approximately 2400 nm output peak wavelength, and an approximately 1 watt (W) output power.

By emitting a mid-infrared laser beam having a high fat or water absorbent property, the emitting unit 113 may minimize an effect on a normal tissue in the body and safely eliminate the abnormal tissue.

The guide beam generating unit 115 may generate a guide beam coupled with the emitted laser beam to visualize the laser beam emitted to the object, thereby verifying a position of the object to which the laser beam is emitted. In this instance, the guide beam generating unit 115 may be disposed in front of or behind the emitting unit 113, and generate a visible laser beam having a wavelength between 532 nm and 633 nm as the guide beam.

When a prism or a chirped mirror is included for dispersion correction, the laser apparatus 100 may emit a tens to hundreds of femtoseconds-pulsed laser beam. Here, femtoseconds may be 10⁻¹⁵ seconds. When a manual optical switch such as the saturated absorber is included, the laser apparatus 100 may emit a pulsed laser beam with a pulse-duration from tens to hundreds of picoseconds. Here, picoseconds may be 10⁻¹² seconds. When an active switch such as a pockels cell for Q switching is included, the laser apparatus 100 may emit a pulsed laser beam with a pulse-duration from tens to hundreds of nanoseconds. Here, the term of nanoseconds may be 10⁻⁹ seconds. Also, the laser apparatus 100 may emit a laser beam having a pulse of which a range varies based on a combination of each element, for example, the prism, the chirped mirror, the manual optical switch, a Q switching, and the like. Thus, the laser apparatus 100 may emit an appropriate pulsed laser beam based on a purpose using an additional element.

By using the guide beam generating unit 115, the laser apparatus 100 may provide an environment enabling an operation to be performed while verifying a position to which a laser beam is emitted in the body. However, the disclosure is not limited thereto. By including a connecting unit (not shown) connected to an endoscope in lieu of the guide beam generating unit 115, the laser apparatus 100 may provide an environment enabling the operation to be precisely performed while visually verifying a fat cell and a tissue cell of the body through the endoscope.

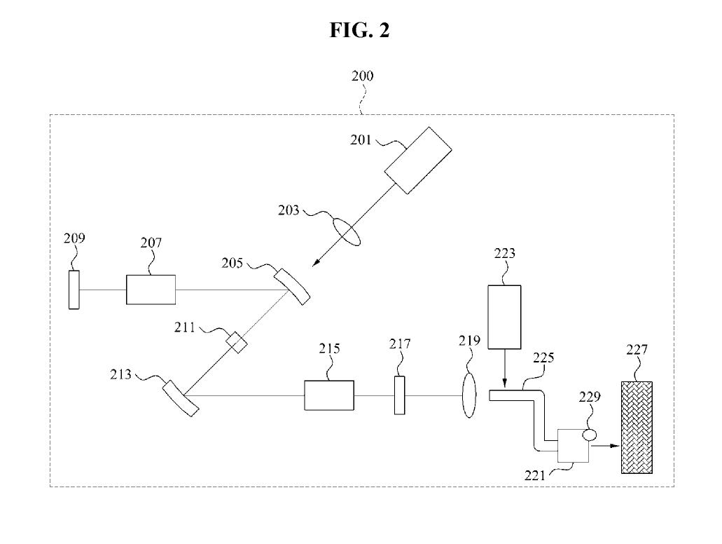

FIG. 2 is a diagram illustrating another example of a laser apparatus 200 according to an embodiment of the present invention.

Referring to FIG. 2, the laser apparatus 200 may include a laser beam generating unit 201, a first lens 203, a first concave mirror 205, an adjusting unit 207, a first parallel mirror 209, a laser crystal 211, a second concave mirror 213, a switch 215, a second parallel mirror 217, a second lens 219, an emitting unit 221, and a guide beam generating unit 223.

The laser beam generating unit 210 may be a pump laser device, for example, an LD, an optical fiber laser device, or a solid-state laser device. The laser beam generating unit 201 may generate a laser beam having a wavelength between 1300 nm and 2100 nm through which an absorbent property of the laser crystal 211 is manifested.

The first lens 203 may be coated for anti-reflection against a wavelength band of the laser beam generated by the laser beam generating unit 201, and may concentrate the generated laser beam. Here, a focal distance of the first lens 201 may be between 25 millimeters (mm) and 150 mm, or at least 150 mm depending on an example.

The first concave mirror 205 may receive the laser beam concentrated by the first lens 203 and transfer the received laser beam to the laser crystal 211 through penetration. Also, the first concave mirror 205 may receive the laser beam of which a wavelength is adjusted, from the laser crystal 211. The received laser beam may be transferred to the first parallel mirror 209 through the adjusting unit 207, and the laser beam reflected from the first parallel mirror 209 may be received by the first concave mirror 205 through the adjusting unit 207. In this instance, the first concave mirror 205 may reflect the received laser beam through the adjusting unit 207 so as to be provided to the laser crystal 211.

The adjusting unit 207 may be, for example, a birefringent filter. The adjusting unit 207 may adjust a peak wavelength of the laser beam received from the first concave mirror 205 based on an input wavelength adjustment value. In this example, the adjusting unit 207 may adjust the wavelength of the laser beam to be within an output range, for example, from 1700 nm to 3500 nm, in the laser crystal 211.

The first parallel mirror 209 may perform high reflection on the laser beam received from the adjusting unit 207 such that approximately 100 percent (%) of the laser beam is reflected. Subsequently, the first parallel mirror 209 may provide the reflected laser beam to the first concave mirror 205 through the adjusting unit 207. Accordingly, the laser beam reflected from the first parallel mirror 209 may be transferred through the laser crystal 211, the second concave mirror 213 and the switch 215, to the second parallel mirror 217 performing a function of an output mirror.

The laser crystal 211 may be disposed between the first concave mirror 205 and the second concave mirror 213. The laser crystal 211 may perform a function to convert the laser beam, for example, a pump beam, generated by the laser beam generating unit 201 into a laser beam of which a wavelength band is between 1700 nm and 3500 nm.

The laser crystal 211 may be a laser crystal doped with, for example, chromium ions, and not be limited in terms of a size and a shape. A host substance for use in the laser crystal 211 may be, for example, a zinc sulfide (ZnS), a zinc selenide (ZnSe), a zinc sulfide-selenium (ZnSSe), and the like.

The laser crystal 211 may be fixed to a holder having a high thermal conductivity. To enhance a thermal conductivity, the laser crystal 211 may be covered with a substance such as In, and fixed to the holder. In this example, the holder may be cooled by air, connected to a chiller to be cooled by water, or connected to a thermoelectric element to be cooled.

The second concave mirror 213 may reflect the laser beam received from the laser crystal 211 and provide the reflected laser beam to the switch 215.

A reflection rate of each of the first parallel mirror 209, the first concave mirror 205, and the second concave mirror 213 may be greater than or equal to 99% in a range from 1700 nm to 3500 nm. To efficiently penetrate the laser beam generated by the laser beam generating unit 201, the first parallel mirror 209, the first concave mirror 205, and the second concave mirror 213 may be coated for anti-reflection against a beam corresponding to the wavelength of the laser beam.

The switch 215 may receive the laser beam output from the second concave mirror 213. In response to a laser mode change request, the switch 215 may transform the received laser beam to one of a continuous laser beam and a pulsed laser beam, which is related to the laser mode change request, and output a result of the transforming.

The second parallel mirror 217 may penetrate the laser beam output from the switch 215. In this example, a penetration rate of the second parallel mirror 217 may be between 0.5% and 20% in a range, for example, from 1700 nm to 3500 nm. The penetration rate may vary based on a doping ratio of Cr²⁺ ions in the laser crystal 211.

The second lens 219 may concentrate the laser beam received from the second parallel mirror 217. The concentrated laser beam may be coupled with an optical fiber 225 and transferred to the emitting unit 221.

The emitting unit 221 may emit the laser beam received from the second lens 219 through the optical fiber 225 to an object 227. The emitting unit 221 may be, for example, the cannula, and may emit a laser beam to an abnormal tissue of a body.

The guide beam generating unit 223 may be disposed between the second lens 219 and the emitting unit 221 and generate a guide beam to visualize the concentrated laser beam, thereby verifying a position to which the laser beam is emitted in the object 227. Here, the guide beam may be, for example, a visible laser beam having a wavelength between 532 nm and 633 nm, and coupled with the concentrated laser beam.

The guide beam generating unit 233 may be disposed between the second lens 219 and the emitting unit 221, and disposed behind the emitting unit 221. The guide beam generating unit 223 may be, a light emitting diode (LED), and disposed on an end (229) of the emitting unit 221.

The laser apparatus 200 may include elements forming a Z-shape, an X-shape, or an L-shape.

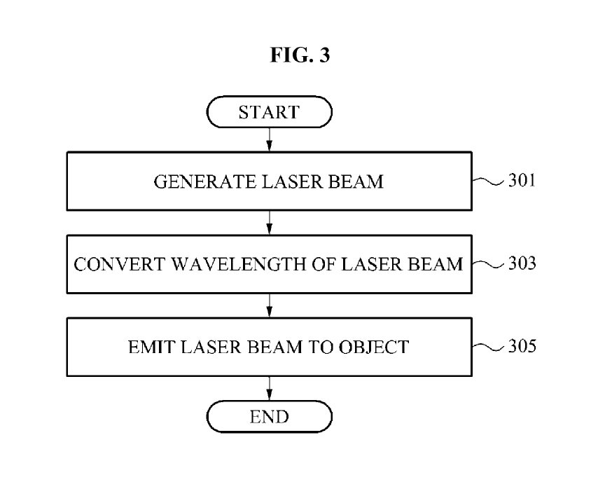

FIG. 3 is a flowchart illustrating a method of operating a laser apparatus according to an embodiment of the present invention.

Referring to FIG. 3, in operation 301, the laser apparatus may generate a laser beam.

The laser apparatus may adjust a wavelength of the generated laser beam based on an input wavelength adjustment value.

In operation 303, the laser apparatus may convert the wavelength of the generated laser beam. In this example, the laser apparatus may convert the wavelength of the generated laser beam to be a set wavelength.

In this example, the laser apparatus may adjust the wavelength of the generated laser beam based on the input wavelength adjustment value.

In response to a laser mode change request, the laser apparatus may transform the laser beam having the converted wavelength, to one of a continuous laser beam and a pulsed laser beam, which is related to the laser mode change request.

In operation 305, the laser apparatus may emit the laser beam to an object. In this example, the laser apparatus may emit a laser beam having a fat or water absorbent property greater than or equal to a set reference value.

The laser apparatus may generate a guide beam coupled with the emitted laser beam to visualize the laser beam.

According to an aspect of the present invention, it is possible to provide a laser apparatus for emitting a mid-infrared laser beam having a high fat or water absorbent property using a chromium ion-doped laser crystal, thereby safely eliminating an abnormal tissue while minimizing an effect on a normal tissue of a body, and an operation method thereof.

According to another aspect of the present invention, it is possible to selectively output a continuous laser beam or a pulsed laser beam or adjust a wavelength of a laser beam in response to a laser mode change request, thereby emitting an appropriate laser beam based on a situation or a purpose.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. A laser apparatus comprising:
a laser beam generating unit to generate a laser beam;
a converting unit to convert a wavelength of the generated laser beam to be a set wavelength; and
an emitting unit to emit the laser beam having the converted wavelength to an object.

2. The apparatus of claim 1, wherein the converting unit comprises a laser crystal doped with chromium ions to output a laser beam having a wavelength between 1700 nanometers (nm) and 3500 nm.

3. The apparatus of claim 1, further comprising:
a switch to transform, in response to a laser mode change request, the laser beam to one of a continuous laser beam and a pulsed laser beam, which is related to the laser mode change request.

4. The apparatus of claim 1, further comprising:
an adjusting unit to adjust the converted wavelength of the laser beam based on an input wavelength adjustment value.

5. The apparatus of claim 1, further comprising:
a guide beam generating unit to generate a guide beam coupled with the emitted laser beam to visualize the laser beam.

6. The apparatus of claim 1, wherein the emitting unit comprises a cannula to emit the laser beam having the converted wavelength, to a body as the object.

7. The apparatus of claim 1, wherein the emitting unit emits the laser beam having a fat or water absorbent property greater than or equal to a set reference value.

8. The apparatus of claim 1, further comprising:
a first mirror to receive the generated laser beam, transfer the received laser beam to the converting unit, transfer the laser beam of which the wavelength is converted by the converting unit to an adjusting unit such that a wavelength of the laser beam is adjusted, reflect the laser beam received from the adjusting unit, and retransfer the reflected laser beam to the converting unit; and
a second mirror to penetrate a portion of the laser beam output from the converting unit, and transfer the penetrated portion of the laser beam to the emitting unit.

9. A laser apparatus comprising:
in response to a laser mode change request,
a converting unit to convert a wavelength of a laser beam generated by a laser beam generating unit; and
a switch to transform the laser beam having the converted wavelength, to one of a continuous laser beam and a pulsed laser beam.

10. The apparatus of claim 9, further comprising:
an adjusting unit to adjust the wavelength of the generated laser beam based on an input wavelength adjustment value.

11. The apparatus of claim 9, further comprising:
a guide beam generating unit to generate a guide beam coupled with the laser beam to visualize the laser beam.

12. A method of operating a laser apparatus, the method comprising:
generating a laser beam;
converting a wavelength of the generated laser beam to be a set wavelength; and
emitting the laser beam having the converted wavelength, to an object.

13. The method of claim 12, further comprising:
transforming, in response to a laser mode change request, the laser beam having the converted wavelength to one of a continuous laser beam and a pulsed laser beam, which is related to the laser mode change request.

14. The method of claim 12, further comprising:
adjusting the converted wavelength of the laser beam based on an input wavelength adjustment value.

15. The method of claim 12, further comprising:
generating a guide beam coupled with the emitted laser beam to visualize the laser beam.

16. The method of claim 12, wherein the emitting comprises emitting the laser beam having a fat or water absorbent property greater than or equal to a set reference value.
